(19) United States
(12) Patent Application Publication
(10) Pub. No.: US 2023/0119560 A1
(43) Pub. Date: Apr. 20, 2023

(54) KIDNEY REGENERATION ACCELERATOR AND PRODUCTION METHOD FOR SAME

(71) Applicants: KEIO UNIVERSITY, Minato-ku (JP); JSR Corporation, Minato-ku (JP)

(72) Inventors: Hiroshi YAGI, Tokyo (JP); Hiroko KUSHIGE, Tokyo (JP); Kohei KURODA, Tokyo (JP); Akiko SATO, Tokyo (JP); Kenichi HAMADA, Tokyo (JP); Yuki AMANO, Tokyo (JP)

(73) Assignees: KEIO UNIVERSITY, Minato-ku (JP); JSR Corporation, Minato-ku (JP)

(21) Appl. No.: 17/792,348

(22) PCT Filed: Jan. 15, 2021
(86) PCT No.: PCT/JP2021/001241

§ 371 Date: Jul. 12, 2022

(30) Foreign Application Priority Data
Jan. 17, 2020 (JP) 2020-006083

(51) Int. Cl. A61L 27/36

(52) U.S. Cl. CPC A61L 27/3604; A61L 27/3679; A61L 27/3691; A61L 2430/26; A61L 2430/40

(57) ABSTRACT

The kidney regeneration accelerator that contains a component obtained by decellularizing a mammalian organ. The production method for a kidney regeneration accelerator that involves decellularizing a mammalian organ to obtain a component that includes an extracellular matrix, freeze drying and then pulverizing the component to obtain a powder, and performing a sterilization treatment on the powder. A pharmaceutical composition for use in treating kidney disease that contains a component obtained by decellularizing a mammalian organ. A treatment method for kidney disease that involves applying a pharmaceutical composition that contains a component obtained by decellularizing a mammalian organ to a site to be treated of the kidney of a human or animal kidney disease patient.

### TECHNICAL FIELD

The present invention relates to a kidney regeneration accelerator and a production method for same.

Priority is claimed on Japanese Patent Application No. 2020-006083 filed Jan. 17, 2020, the content of which is incorporated herein by reference.

### BACKGROUND ART

While research on the regeneration of thin layer tissues such as skin, gastrointestinal mucosa, and cornea, and tissues with relatively simple structures and functions such as bone and a soft tissue has acceleratingly progressed with the recent development of the regenerative medical field, organ-based research and development is lagging behind. The reason is that it is still difficult to understand and reproduce the extremely complicated structure and function of three-dimensional organs. The development of functional organ regeneration technology that can be a radical treatment for the failure of parenchymal organ such as liver, kidney, and pancreas, which have high social needs, is still in the middle of the process. Until now, various technological developments have been carried out to realize the regeneration of three-dimensional organs.

On the other hand, the importance of the role of extracellular matrix (ECM) in the structure and function of organs has been strongly recognized in recent years. Actually, ECM plays a central role from the stage of fetal development. ECM is composed of collagen, laminin, fibronectin, glycosaminoglycan (GAG), or the like, and affects all the basic functions for proliferation, stabilization, differentiation, and the like of cells themselves, as a scaffold structure that dynamically controls the pericellular environment by production/absorption.

Regeneration of organ structure requires 1) appropriate ECM, 2) continuous three-dimensional structure from microstructure to large blood vessels, and 3) sufficient supply of cells. In order to realize the regeneration of such complicated three-dimensional organs, Ott et al. has reported a method to apply the decellularized organ skeleton of the parenchymal organ itself to regenerative medicine in 2008, ahead of others in the world (for example, see Non Patent Document 1). In this method, all cells are removed from a living tissue by various methods, and the skeleton of ECM, which is a remaining fibrous protein, is used for tissue regeneration. Actually, a decellularized tissue using human skin (Alloderm (registered trademark)) and a decellularized tissue using pig heart valve (Hancock (registered trademark)) obtained by the same method have been already commercialized and clinically applied as a medical material.

In addition, a technique has been developed in which a substance obtained by pulverizing a decellularized tissue is used. For example, Patent Document 1 discloses a hydrogel containing a pulverized product of a decellularized tissue obtained by decellularizing an animal-derived backboard tissue for the purpose of improving adhesiveness to a target site, and the like, fibrinogen, and thrombin.

### PRIOR ART LITERATURE

### Patent Document

Patent Document 1: PCT International Publication No. WO2014/181886

### Non Patent Document

Non Patent Document 1: Ott H C et al., “Perfusion-decellularized matrix: using nature’s platform to engineer a bioartificial heart.”, Nat Med., Vol. 14, Issu 2, pp. 213-21, 2008.

### DISCLOSURE OF INVENTION

### Problems to Be Solved by the Invention

The present invention provides a novel kidney regeneration accelerator effective for the treatment of kidney disease and a production method for same.

### Means for Solving the Problems

That is, the present invention includes the following aspects.

A kidney regeneration accelerator containing a component obtained by decellularizing an organ of a mammal.

The kidney regeneration accelerator according to (1), which is one selected from the group consisting of a solution, a dispersion and a gel containing the component.

The kidney regeneration accelerator according to (2), in which a concentration of the component is 5 mg/mL or more and 25 mg/mL or less in the total volume of the kidney regeneration accelerator.

The kidney regeneration accelerator according to (2) or (3), which has a viscosity of 10 mPa·s or more and 1000 mPa·s or less.

The kidney regeneration accelerator according to (1), which is a powder.

The kidney regeneration accelerator according to any one of (1) to (5), in which the component is obtained by decellularization including hydrostatic pressure treatment.

The kidney regeneration accelerator according to any one of (1) to (6), in which the organ is one or more organs selected from the group consisting of a liver, a kidney, a spleen, a lung, a pancreas, an intestine, and a blood vessel.

A method of decellularizing an organ of a mammal to prepare a component containing an extracellular matrix, the method including:
performing hydrostatic pressure treatment on the organ or a fragment of the organ; and
perfusing the organ with water or stirring the fragment in water,
in which the hydrostatic pressure treatment includes
pressurization including a positive pressure change in which an absolute value of an amount of the change is 100 MPa or more, and
decompression including a negative pressure change in which an absolute value of an amount of the change is 50 Mpa or more each of which is alternately performed two times or more, and
the pressurization and the decompression are both performed at a pressure of 0 MpaG or more.

The method according to (8), in which the hydrostatic pressure treatment is performed in a state where the organ is in contact with a surfactant or a solution containing the surfactant.

A production method of a kidney regeneration accelerator, the method including:
decellularizing an organ of a mammal to obtain a component containing an extracellular matrix;
freeze drying and pulverizing the component to obtain a powder; and
performing sterilization treatment on the powder.

The production method for a kidney regeneration accelerator according to (10), in which the freeze drying is performed two times or more.

The production method for a kidney regeneration accelerator according to (10), in which the obtaining the component containing the extracellular matrix includes:
performing hydrostatic pressure treatment on the organ or a fragment of the organ; and
perfusing the organ with water or stirring the fragment in water.

The production method for a kidney regeneration accelerator according to (12), in which the hydrostatic pressure treatment includes
pressurization including a positive pressure change in which an absolute value of an amount of the change is 100 Mpa or more, and
decompression including a negative pressure change in which an absolute value of an amount of the change is 50 Mpa or more each of which is alternately performed two times or more, and
the pressurization and the decompression are both performed at a pressure of 0 MpaG or more.

The production method for a kidney regeneration accelerator according to (12) or (13), in which the hydrostatic pressure treatment is performed in a state where the organ is in contact with a surfactant or a solution containing the surfactant.

A pharmaceutical composition for use in treating kidney disease, which containing a component obtained by decellularizing an organ of a mammal.

The pharmaceutical composition according to (15), which is one selected from the group consisting of a solution, a dispersion and a gel.

The pharmaceutical composition according to (15), which is a powder.

The pharmaceutical composition according to any one of (15) to (17), in which the mammal is a mammal other than a human.

The pharmaceutical composition according to any one of (15) to (18), in which the organ is one or more organs selected from the group consisting of liver, kidney, spleen, lung, pancreas, intestine, and a blood vessel.

A treatment method for kidney disease, which includes applying a pharmaceutical composition containing a component obtained by decellularizing an organ of a mammal to a site to be treated of a kidney of a human or animal kidney disease patient.

### EFFECTS OF THE INVENTION

According to the kidney regeneration accelerator and the production method for same, of the above aspect, it is possible to provide a novel kidney regeneration accelerator effective for the treatment of kidney disease and a production method for same.

### BRIEF DESCRIPTION OF DRAWINGS

FIG. 1 is a hematoxylin-eosin (HE) stained image of a skin tissue section in which dECM gel or GelMA hydrogel has been embedded in Test Example 1.

FIG. 2 is an observation image of the skin tissue section in which dECM gel or GelMA hydrogel has been embedded in Test Example 1.

FIG. 3 is a cryo-scanning electron microscope (cryo-SEM) image of the L-dECM gel prepared in Example 3.

FIG. 4 is an observation image (left) and a HE stained image (right) of an excised section of kidney in Test Example 2.

FIG. 5 is an immunostaining image of the excised section of kidney in Test Example 2.

FIG. 6 is an observation image (left) and an HE stained image (right) of an excised section of kidney in Test Example 3.

FIG. 7 is an immunostaining image of the excised section of kidney in Test Example 3.

FIG. 8 is an HE stained image of an excised section of kidney (8 mg/mL K-dECM gel injection) in Test Example 4.

FIG. 9 is an HE stained image of an excised section of kidney (16 mg/mL K-dECM gel injection) in Test Example 4.

FIG. 10 is cryo-SEM images of K-dECM gels having different concentrations (8 mg/mL and 16 mg/mL) prepared in Example 5.

FIG. 11 is cryo-SEM images of K-dECM gels prepared in Example 6 in which the various amounts of proteoglycan were added.

### EMBODIMENTS FOR CARRYING OUT THE INVENTION

Hereinafter, the present invention will be described in more detail with reference to embodiments, but the present invention is not limited to the following embodiments.

### Kidney Regeneration Accelerator

The kidney regeneration accelerator according to one embodiment of the present invention (hereinafter, it may be simply referred to as “a kidney regeneration accelerator of the present embodiment”) contains a component obtained by decellularizing an organ of a mammal (hereinafter, it may be simply referred to as “a component”).

Tests with cell-loaded transplantation tissues in the kidney have already been performed using different techniques prior to the use of decellularized skeleton. One of the transplantation tissues is a tubular hollow fiber-type structure in which distal renal tubular epithelial cells are cultured and engrafted, and it has been, for example, reported that dogs with uremia were actually cured. However, since the fiber-type structure has structural problems such as blood perfusion at the time of embedding in the body, it has been pointed out that a material that can be transplanted and is efficiently adapted to the living body is necessary.

On the other hand, the inventors have been first revealed that when the kidney regeneration accelerator of the present embodiment is injected into the kidney defective part of a rat or a pig, cell infiltration at the injected part and further regeneration of renal tubules and glomeruli are observed, and the kidney can be regenerated efficiently.

The kidney regeneration accelerator of the present embodiment contains a component obtained by decellularizing an organ of a mammal, so that rejection reaction is unlikely to occur after filling, and regeneration and treatment of a defective site or damaged site of the kidney can be performed.

In the present specification, the “component obtained by decellularizing an organ of a mammal” refers to a component obtained by decellularizing at least a part of cells contained in an organ of a mammal.

The form of the kidney regeneration accelerator of the present embodiment is not particularly limited, and may be, for example, a solution, a dispersion, a sol or a gel, or a powder. Above all, it is preferably that the kidney regeneration accelerator is in the form of a solution or dispersion, and is gel form after application to the treatment target site such that the kidney regeneration accelerator is retained at the target site when using by immediately filling, adhering, or applying to the site to be treated (defective site or damaged site) of the kidney. The term “gel form” as used herein means a state in which the extracellular matrix (ECM) contained in the above component has a network structure by chemical bonding and holds a solvent in the network, and the gel form kidney regeneration accelerator is in a state of having viscosity and losing fluidity, or in a state in which the fluidity is greatly reduced. On the other hand, from the viewpoint of stability during transportation and storability, it is preferably powdery. The powdery kidney regeneration accelerator can be made into a gel form by mixing with a solvent (dispersion medium), and can be used by filling the defective part or damaged part of the kidney.

The kidney regeneration accelerator of the present embodiment is suitably used in treating kidney disease. That is, the kidney regeneration accelerator of the present embodiment can also be said to be a pharmaceutical composition for use in treating kidney disease.

The kidney disease to which the treatment with the kidney regeneration accelerator of the present embodiment is applied is not particularly limited as long as it is a kidney disease accompanied by a kidney defect due to the disease or a kidney defect due to surgical treatment, and examples of the kidney disease include polycystic kidney, nephritis, renal parenchymal tumor (renal cell cancer), renal pelvis tumor (renal pelvis cancer), diabetic nephropathy, chronic kidney disease, collagen disease-derived nephropathy, nephrosclerosis, pyelitis, renal abscess, pyonephrosis, perinephritis, perinephritic abscess.

The animal to be treated is preferably a mammal. Examples of the mammal include a human, a monkey, a marmoset, a cow, a horse, a sheep, a pig, a goat, a deer, an alpaca, a dog, a cat, a rabbit, a hamster, a guinea pig, a rat, a mouse and the like. Above all, a human is preferable.

### Component Obtained by Decellularizing Organ of Mammal

The component is obtained by decellularizing an organ of a mammal and contains ECM as its main component. The preparation method for the component will be described later in the production method for a kidney regeneration accelerator.

In the present specification, the term “extracellular matrix (ECM)” means a substance that is found between cells of animal tissue and that functions as a structural element within the tissue. ECM contains a mixture of proteins and polysaccharides secreted by cells. Specifically, ECM is composed of collagen, laminin, fibronectin, glycosaminoglycan (GAG), or the like, and is particularly rich in collagen. The types of components and their composition ratios are different depending on the types of organs from which they are derived.

The organ from which the above component is derived is preferably an organ derived from the endoderm as well as the kidney to be filled, and examples of the organ include a liver, a kidney, a spleen, a lung, a pancreas, an intestine (small intestine, large intestine, and the like), and a blood vessel.

The mammal is preferably a mammal other than human. Examples of a mammal other than human include a monkey, a marmoset, a cow, a horse, a camel, a llama, a donkey, a yak, a sheep, a pig, a goat, a deer, an alpaca, a dog, a raccoon dog, a Japanese mink, a fox, a cat, a rabbit, a hamster, a guinea pig, a rat, a mouse, a squirrel, and a raccoon. Above all, in view of the stable availability, a domestic mammal is preferable, and a pig or a rat is particularly preferable.

The concentration of the above component in the kidney regeneration accelerator of the present embodiment can be appropriately changed depending on the size of the defective part or damaged part of the kidney and the type of filling method of the kidney regeneration accelerator. For example, with respect to the total volume of the kidney regeneration accelerator, the concentration of the component can be about 0.1 mg/mL or more and 100 mg/mL or less, and is preferably 1 mg/mL or more and 50 mg/mL or less, more preferably 5 mg/mL or more and 25 mg/mL or less, and still more preferably 8 mg/mL or more and 16 mg/mL or less.

### Solvent (Dispersion Medium) Component

The kidney regeneration accelerator of the present embodiment may further contain a solvent component or a dispersion medium component. Hereinafter, in the present specification, both a solvent and a dispersion medium are referred to simply as “solvent”, unless otherwise indicated.

As the solvent component, any solvent may be used as long as it is biocompatible and does not exhibit cytotoxicity when filled into the defective part or damaged part of the kidney, and examples of the solvent component include water and a physiological aqueous solution. The physiological aqueous solution may be an isotonic aqueous solution in which the salt concentration, sugar concentration, and the like are adjusted with sodium, potassium, or the like such that the osmotic pressure is substantially the same as that of the body fluid or cell fluid. Examples of the physiological aqueous solution include physiological saline, physiological saline having a buffering effect (phosphate buffered saline [PBS], tris buffered saline [TBS], HEPES buffered saline, and the like), Ringer’s solution, lactate Ringer’s solution, acetate Ringer’s solution, bicarbonate Ringer’s solution, 5% glucose aqueous solution and the like, and the physiological aqueous solution is not limited thereto. Above all, PBS is preferable.

### Other Component

The kidney regeneration accelerator of the present embodiment may contain other components in addition to the above component.

Examples of other components include therapeutic agents and bioactive substances that promote the regeneration of defective part or damaged part of the kidney; antibiotics, antibacterial agents, antiviral agents, and the like for preventing infectious diseases and the like.

### Viscosity Adjustment

In a case where the kidney regeneration accelerator of the present embodiment is gel form, the kidney regeneration effect can be further improved by controlling the viscosity within an appropriate range.

The specific viscosity of the gel form kidney regeneration accelerator is preferably 10 mPa·s or more and 1000 mPa·s or less, more preferably 20 mPa·s or more and 400 mPa·s or less, and particularly preferably 30 mPa·s or more and 350 mPa·s or less. In a case of where the viscosity is not less than the above lower limit value, the kidney regeneration accelerator can be retained at the site to be treated for a longer period of time while the good infiltration property of the cells is maintained. On the other hand, in a case of where the viscosity is not more than the above upper limit value, it is possible to improve the infiltration property of the cells while the kidney regeneration accelerator is retained at the site to be treated.

The viscosity of the gel form kidney regeneration accelerator can be adjusted by changing the decomposition treatment time with pepsin as described in the mixing step of the production method described later. For example, the treatment time is preferably 48 hours or more and 168 hours or less, and more preferably 48 hours or more and 96 hours or less.

Alternatively, the viscosity can be adjusted by adjusting the amount of the solvent (dispersion medium) component to be added and adjusting the concentration of the above component in the kidney regeneration accelerator of the present embodiment.

Alternatively, the viscosity can be adjusted by adding a thickening agent to the gel form kidney regeneration accelerator after the pepsin treatment. Examples of thickening agent include polyethylene glycol, gelatin, hyaluronic acid, proteoglycan, and various peptides.

In a case where proteoglycan is used as a thickening agent, with respect to the total volume of the kidney regeneration accelerator, the concentration of proteoglycan can be, for example, 0.01 µg/mL or more and 20 µg/mL or less, and is preferably 0.05 µg/mL or more and 15 µg/mL or less, and more preferably 0.06 µg/mL or more and 12 µg/mL or less.

### Production Method of Kidney Regeneration Accelerator

The production method for a kidney regeneration accelerator of the present embodiment (hereinafter, may be simply referred to as “the production method of the present embodiment”) includes
decellularizing an organ of a mammal to obtain a component containing ECM (hereinafter, may be referred to as “a decellularization step”),
after freeze drying of the component, pulverizing the component to obtain a powder (hereinafter, may be referred to as “a powdering step”), and
performing the sterilization treatment of the powder (hereinafter, may be referred to as “a sterilization step”).

In the production method of the present embodiment, the “component containing ECM” is synonymous with the “component obtained by decellularizing an organ of a mammal” mentioned above.

### Decellularization Step

In the decellularization step, an organ of a mammal is decellularized to obtain a component containing ECM. The decellularization step can also be said to be a method of decellularizing an organ of a mammal to prepare a component containing an extracellular matrix. Decellularization is not particularly limited as long as it is a method for removing animal-derived cells, viruses, and bacteria (hereinafter, may be collectively referred to as “cells and the like”). Examples of the decellularization method include surfactant treatment, enzyme treatment, osmotic pressure treatment, freezing-thawing treatment, hydrostatic pressure treatment, perfusion treatment (step of perfusing water or the like inside an organ), and stirring treatment in a liquid (step of stirring the organ as it is or shredded in water or the like). These treatments can be appropriately selected according to the type of a mammal or an organ, and can be used in combination as necessary. Above all, surfactant treatment, hydrostatic pressure treatment, perfusion treatment, or stirring treatment in a liquid, or a combination thereof is preferable. The removal of cells and the like is promoted, for example, by using a surfactant in combination with a hydrostatic pressure treatment, a perfusion treatment, or a stirring treatment in a liquid. Alternatively, by performing the hydrostatic pressure treatment before the perfusion treatment or the stirring treatment in the liquid, the amount of the surfactant used can be reduced or eliminated, and a surfactant having a smaller effect on ECM can be selected, in addition to promoting the removal of cells and the like. This combination is effective when it is desired to reduce the amount of the surfactant contained in the kidney regeneration accelerator, thus it is a particularly preferable.

The lower limit of the pressure applied in the hydrostatic pressure treatment is generally larger than 0 MPaG, and is preferably 10 MPaG or more, more preferably 50 MPaG or more, still more preferably 150 MPaG or more. The upper limit is generally 1000 MPaG, and is preferably 750 MPaG or less, and more preferably 500 MPaG or less. Pressurization may be performed once, or pressurization and decompression may be divided and alternately repeated in multiple times (two times or more).

Pressurization here is a hydrostatic pressure treatment including a positive pressure change in which the absolute value of the change amount is 100 MPa or more, while decompression is a hydrostatic pressure treatment including a negative pressure change in which the absolute value of the change amount is 50 MPa or more. In addition, these pressurization and decompression are both preferably performed at a pressure of 0 MPaG or more.

The conditions for decellularization can be appropriately selected depending on the type of a mammal or an organ. Specifically, examples of the conditions include the condition shown in the Examples described later.

In a case where the perfusion treatment or the stirring treatment in a liquid is performed, these treatments may be performed alone or in combination with the hydrostatic pressure treatment. By performing a perfusion treatment or a stirring treatment in a liquid after the hydrostatic pressure treatment, the decellularization treatment can be performed more efficiently. The perfusion treatment and the stirring treatment in a liquid are generally performed using water. The water in this case may contain a surfactant. The surfactant is not particularly limited, and examples thereof include an ionic surfactant and a nonionic surfactant. One of these may be used alone or a combination of two or more may be used.

The perfusion treatment can be performed using a known perfusion device. By performing the stirring treatment in a liquid in a state where the organ is shredded, the decellularization treatment can be performed more efficiently.

Examples of the ionic surfactant include sodium fatty acid, potassium fatty acid, sodium alpha-sulfofatty acid ester, sodium linear alkylbenzene sulfonate, sodium alkyl sulfate, sodium alkyl ether sulfate, sodium alpha-olefin sulfonate, 3-[(3-Cholamidopropyl)dimethylammonio]propane sulfonate (CHAPS). One of these may be used alone or a combination of two or more may be used. Above all, sodium fatty acid or CHAPS is preferable, and sodium dodecyl sulfate (SDS) or CHAPS is more preferable.

Examples of the non-ionic surfactant include alkyl glycoside, alkyl polyoxyethylene ether (Brij series or the like), octylphenol ethoxylate (Triton X series, Igepal CA series, Nonidet P series, Nikkol OP series, or the like), polysorbates (Tween series such as Tween 20, or the like), sorbitan fatty acid ester, polyoxyethylene fatty acid ester, alkyl maltoside, sucrose fatty acid ester, glycoside fatty acid ester, glycerin fatty acid ester, propylene glycol fatty acid ester, fatty acid monoglyceride, and the like. One of these may be used alone or a combination of two or more may be used.

The decellularization step can include a step of perfusing water in the organ to wash (hereinafter, may be referred to as “a washing step”) before the decellularization. The perfusion of water into the organ in the washing step can be performed using the same device as the perfusion treatment mentioned above.

In addition, the decellularization step can further include a step of shredding an organ derived from a mammal (hereinafter, may be referred to as “a shredding step”) before the decellularization. As a result, decellularization of the organ can be performed more efficiently. Shredding of the organ can be performed using, for example, a known shredding device (cutter) or the like. The shredding step is preferably performed after the washing step.

The decellularization step can further include a step of washing the component obtained by decellularizing the organ. The method for washing the component can be appropriately selected depending on the type of the decellularization method. Examples of the method of washing include a method of immersing in a washing liquid, a method of irradiating with microwaves, and the like.

### Powdering Step

In the powdering step, the component obtained by decellularizing the organ is powdered. Generally, the component is freeze-dried and then pulverized to obtain a powder. The number of times of freeze drying may be once or multiple times of two or more times, and the freeze drying is performed preferably multiple times of two times or more, more preferably two or three times, and still more preferably two times.

The powdering step can further include a step of shredding the component before freeze drying. As a result, the component can be pulverized more efficiently.

Examples of the method of powdering include a ball mill, a bead mill, a colloidal mill, a conical mill, a disc mill, an edge mill, a powdering mill, a hammer mill, a pellet mill, a cutting mill, a roller mill, and a jet mill.

In addition, the powdering step can further include a step of sieving the obtained powder based on a particle size.

### Sterilization Step

The powder obtained in the above powdering step is usually used after undergoing one or more sterilization steps. As the sterilization step, known methods such as high pressure steam sterilization, dry heat sterilization, ethylene oxide (EO) gas sterilization, low temperature gas plasma sterilization, and gamma ray sterilization can be used. Among these, ethylene oxide gas sterilization or gamma ray sterilization is preferable, and gamma ray sterilization is particularly preferable, in order to minimize the denaturation and deterioration of the kidney regeneration accelerator. As for the timing of the sterilization step, the sterilization step is preferably performed in a state where the kidney regeneration accelerator is a powder, similarly in order to minimize the denaturation and deterioration of the kidney regeneration accelerator.

The production method of the present embodiment can include other steps in addition to the decellularization step, the powdering step, and the sterilization step.

Examples of other steps include a mixing step and a drying step.

### Mixing Step

In the mixing step, a solvent is mixed with the powder obtained in the above powdering step to obtain a gel form kidney regeneration accelerator. The above powder can be used as it is as a powdery kidney regeneration accelerator, but in a case where repowdering is performed after undergoing mixing step, the gelled state can be confirmed and the characteristics in the gel state can also be confirmed. Examples of the type of solvent include the same as those exemplified in the above kidney regeneration accelerator. The mixing method is not particularly limited and can be performed using a known stirrer or the like.

The mixing ratio (solvent/powder) of the solvent to the powder may be any ratio as long as a viscosity is suitable for the filling method into the defective part of the kidney, and is, for example, about 10/1 or more and 10000/1 or less, and preferably 30/1 or more and 100/1 or less, in terms of mass ratio.

The mixing step can further include a step of adding pepsin to the mixture of the powder and the solvent to perform the decomposition treatment. As a result, the protein component contained in the powder can be decomposed more finely, and the solubility and dispersibility can be enhanced to obtain a more homogeneous kidney regeneration accelerator in solution or gel form. In addition, in a case where this step is performed, the mixing step preferably further includes a step of adding an alkali or an acid to inactivate the enzyme after the decomposition treatment with pepsin.

### Drying Step

In the drying step, the gel form kidney regeneration accelerator obtained in the above mixing step is dried and then pulverized to obtain a powdery kidney regeneration accelerator. The gel obtained in the above mixing step can be used as a gel form kidney regeneration accelerator, but in a case of undergoing the drying step, storage and transportation are facilitated and control of the characteristics of the powder is also facilitated. In particular, in a case where treatment with an enzyme such as pepsin is performed, the viscosity may decrease over time in the gel state even if the inactivation treatment is performed. Therefore, preferably, the gel form kidney regeneration accelerator obtained in the above mixing step is dried and powdered again, and the powdered product is used as the powdery kidney regeneration accelerator, or pure water, physiological saline, or the like is added to the powdered product to use as gel form kidney regeneration accelerator.

The drying method is not particularly limited, and examples thereof include known drying methods such as hot air drying, vacuum drying, steam drying, suction drying, and freeze drying. Above all, freeze drying is preferable because denaturation of various components (particularly protein components) contained in the above component can be suppressed. The number of times of freeze drying may be once or multiple times of two or more times, and the freeze drying is performed preferably multiple times of two times or more, more preferably two or three times, and still more preferably two times.

Examples of the pulverization method include the same methods as methods exemplified in the above powdering step.

The powdery kidney regeneration accelerator obtained in this step can be filled into the site to be treated of the kidney by appropriately mixing with a solvent and regelling (rehydration). The amount of the powdery kidney regeneration accelerator used can be appropriately adjusted in consideration of the size of the site to be treated of the kidney to be filled, the viscosity according to the type of filling method, and the like.

Examples of the solvent include the same as those exemplified in the above kidney regeneration accelerator. The amount of the solvent added can be appropriately adjusted in consideration of the size of the site to be treated of the kidney to be filled, the viscosity according to the type of the filling method, and the like.

The mixing ratio (solvent/powdery material) of the solvent to the powdery kidney regeneration accelerator may be any ratio as long as a viscosity is according to the type of filling method, and is, for example, about 20/1 or more and 20000/1 or less in terms of mass ratio.

### Other Embodiments

In one embodiment, the present invention provides a method for treating kidney disease in which a pharmaceutical composition (the above kidney regeneration accelerator) containing a component obtained by decellularizing an organ of a mammal is filled into a site to be treated on a kidney of a human or animal kidney disease patient. Examples of the kidney disease include those similar to those provided as exemplary examples in the above kidney regeneration accelerator. Examples of the human or animal patient include an animal similar to the animal to be treated provided as an exemplary example in the above kidney regeneration accelerator. Examples of the site to be treated of the kidney include a site in which a part of the kidney is lost due to surgical treatment, and a site in which the kidney is damaged due to a kidney disease.

In the method for treating kidney disease, the filling amount of the above kidney regeneration accelerator and the effective amount of the component contained in the kidney regeneration accelerator can be appropriately adjusted in consideration of the size of the site to be treated of the kidney to be filled and the viscosity according to the type of the filling method, and the like.

Examples of the filling method of the above kidney regeneration accelerator include a method of directly filling the site to be treated of the kidney, for example, using a catheter, a syringe, or the like by laparoscopic surgery or robotic surgery. Alternatively, examples thereof include a method of filling the site to be treated of the kidney using a urethral catheter.

In one embodiment, the present invention provides a pharmaceutical composition for use in treating kidney disease, which contains a component obtained by decellularizing an organ of a mammal. Examples of the kidney disease include those similar to those provided as exemplary examples in the above kidney regeneration accelerator.

### EXAMPLES

Hereinafter, the present invention will be described with reference to Examples, but the present invention is not limited to the following Examples.

### Example 1

### Preparation of Kidney Regeneration Accelerator Containing Decellularized Component Derived from Liver (dECM Gel)

### Collection And Storage Of Pig Liver

After intravenous injection of heparin (5000 IU) into a pig (Göttingen minipig), the liver was mobilized and the gallbladder was resected. Subsequently, the bile duct, the hepatic artery, and the lower inferior vena cava were ligated, and then the liver was excised. The portal vein and the upper inferior vena cava were cannulated, and perfusion with physiological saline from the portal vein was performed until blood was no longer drained. After the perfusion, the excised liver was freeze-preserved at -80° C. in a state of being immersed in physiological saline.

### Decellularization by Perfusion

The freeze-preserved liver was slowly thawed at 4° C. It was completely thawed in about 3 days. Subsequently, the thawed liver was perfused with phosphate buffered saline (PBS) from the portal vein for 1 day until the waste liquid became clear (flow rate of 70 mL/min or more and 100 mL/min or less). Subsequently, 0.5 w/v% sodium dodecyl sulfate (SDS) aqueous solution perfusion from the portal vein was performed for 1 day. The perfusion was started at a flow rate of 70 mL/min, and the flow rate was adjusted to be 70 mL/min or more and 320 mL/min or less depending on the degree of cell shedding. In many cases, 0.5 mass% SDS aqueous solution perfusion of about 60 L or more and 80 L or less was performed at 70 mL/min or more and 120 mL/min or less. Subsequently, 100 mL of Triton-X100 (trade name, manufactured by Dow Chemical Co., Ltd.) (final concentration of 0.5 v/v%), 10 g of glycol ether diamine tetraacetic acid (EGTA) (final concentration of 0.05 w/v%), 10 g of sodium azide (final concentration of 0.05 w/v%), and 24 g of amphoteric surfactant (CHAPS) (final concentration of 2 mM) were dissolved in 20 L of PBS. The perfusion with the prepared solution from the portal vein was performed at a flow rate of 150 mL/min. After perfusing the first 10 L, the remaining 10 L was circulated (about 6 hours). Subsequently, perfusions with 500 mL of PBS containing 0.3 mg/mL colistin and then 500 mL of PBS containing 0.2 mg/mL gentamicin were performed to obtain decellularized pig liver tissue (dECM).

### Preparation of dECM Gel

Subsequently, the decellularized pig liver was shredded and then lyophilized for 3 days. Subsequently, the decellularized pig liver shredded product in a dried state was further shredded with a rotary knife mill. Subsequently, 100 mg of pepsin (2000 U/mg or more and 3000 U/mg or less) and 100 mL of 0.01 M HCl were added to 1 g of the re-shredded product, the mixture was stirred at 25° C. for 72 hours, and the re-shredded product was dissolved to obtain a gelled product. Subsequently, 0.1 M NaOH (⅒ amount of gelled product by mass ratio) and 10× PBS (pH 7.4) (⅑ amount of gelled product by mass ratio) were added to the gelled product at 4° C. to inactivate pepsin. The volume and concentration of the dECM were adjusted, and then the dECM gel was stored at 4° C.

### Preparation of Gel Disk for Subcutaneous Embedding

The dECM gel obtained above was injected into a mold. After allowing to stand at 37° C. for 30 minutes, die cutting with a biopsy trepan was performed to prepare a gel disk having a height of about 1 mm and a diameter of about 5 mm.

### Comparative Example 1

### Preparation of Gelatin Methacrylate (GelMA) Hydrogel for Subcutaneous Embedding

100 mg of GelMA was weighed in a 10 mL screw tube bottle, 1.9 g of 1× PBS was added thereto, and the GelMA was dissolved by heating in a water bath at 50° C. 8 mg of photoinitiator Irgacure 2959 (trade name, manufactured by BASF, hereinafter, may be abbreviated as “I2959”) was added and dissolved. After confirming that all of them had been dissolved, the dissolved mixture was sucked into a sterilized disposable syringe and passed through a sterilization filter to sterilize. The required amount of solution was pipetted and injected into the mold. Ultraviolet rays (UVA) were irradiated at 7 mW/cm² for 60 seconds. After allowing to stand for 1 hour, die cutting with a biopsy trepan was performed to prepare a gel disk having a height of about 1 mm and a diameter of about 5 mm.

### Test Example 1

### Rat Subcutaneous Embedding Test of dECM Gel and GelMA

Rats (LEW/CrlCrlj, 10 weeks old, female) were used as experimental animals. Rats were retained in the prone position under isoflurane anesthesia. Subsequently, the dorsal skin was incised by about 5 mm, and the subcutaneous tissue was bluntly peeled from the incision to prepare a subcutaneous pocket having a diameter of about 1 cm. Subsequently, the dECM gel disk prepared in Example 1 or the GelMA gel disk prepared in Comparative Example 1 was inserted into the pocket while being careful not to lose its shape. Subsequently, the skin was closed with a 4-0 nylon thread and the rats were awakened from anesthesia. One week after the subcutaneous embedding, the embedded part including the skin was resected under isoflurane anesthesia and fixed by immersion in PBS containing 4 v/v% paraformaldehyde (PFA). After the embedded part was excised, the animals were promptly euthanized according to a conventional method.

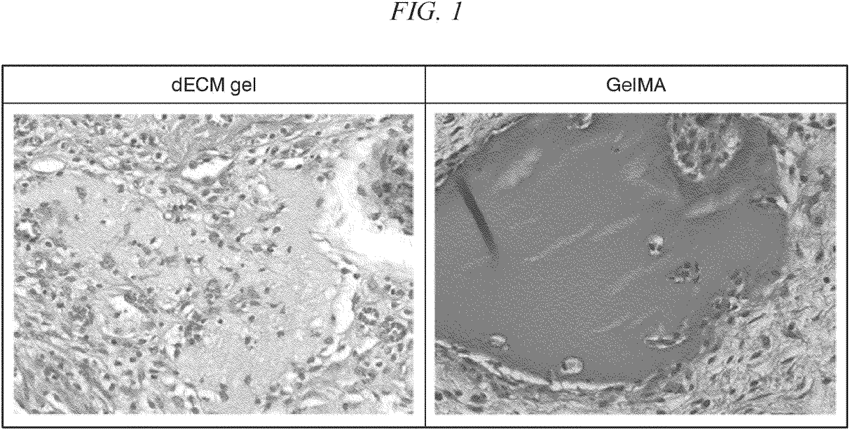

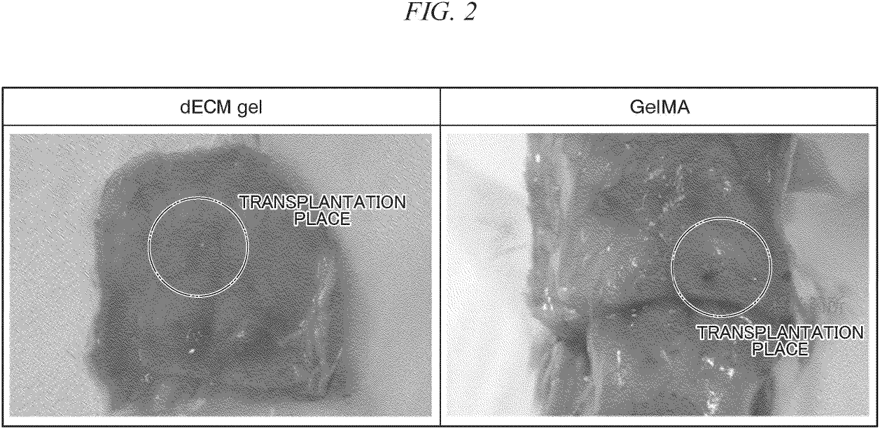

Subsequently, the fixed tissue was shredded to prepare a paraffin-embedded block. Subsequently, the block was sliced to a thickness of about 1 µm to prepare a pathological section. Hematoxylin-eosin (HE) staining was performed according to a conventional method to observe and obtain the images. FIG. 1 is an HE stained image of a pathological section. FIG. 2 is an observation image immediately after the excision of the embedded part.

As shown in FIG. 1, in the dECM gel, remarkable infiltration of cells (vascular endothelial cells and fibroblasts) was observed as compared with the GelMA hydrogel. Furthermore, as shown in FIG. 2, it was confirmed that in the dECM gel, the embedded dECM gel was more integrated with the tissue as compared with the GelMA hydrogel.

### Example 2

### Preparation of Kidney Regeneration Accelerator Containing Decellularized Component Derived from Kidney (K-dECM Gel)

### Collection and Storage of Pig Kidney

After intravenous injection of heparin (5000 IU) into a pig (Göttingen minipig), the renal artery and renal vein were secured and kidney was excised. The outer needle of the 18 G Surflo indwelling needle was cannulated in the renal artery. Subsequently, perfusion with physiological saline from the renal artery was performed until blood was no longer drained. After the perfusion, the excised kidney was freeze-preserved at -80° C. in a state of being immersed in physiological saline.

### Decellularization by Stirring in Liquid

The freeze-preserved kidney was slowly thawed at 4° C. Subsequently, the thawed kidney was shredded into 5 mm square pieces and stirred in PBS for about 24 hours. Subsequently, it was stirred in a 0.5 w/v% SDS aqueous solution for about 18 hours. Subsequently, it was stirred in an aqueous solution containing 0.5 v/v% Triton-X100, 0.05 w/v% EGTA, 0.05 w/v% sodium azide, and 2 mM CHAPS for about 12 hours. Subsequently, it was stirred in PBS for about 60 hours. Subsequently, it was stirred in PBS containing 25 µg/mL Antibiotic Antimycotic Solution (hereinafter, may be abbreviated as “Anti-Anti”) and 0.3 mg/mL colistin for about 1 hour. Subsequently, it was stirred in PBS containing 25 µg/mL Anti-Anti, 1 w/v% gentamicin for about 1 hour to obtain a decellularized pig kidney shredded product. Subsequently, the decellularized pig kidney shredded product was lightly dehydrated by centrifugation, frozen at -80° C., and then freeze-dried. Subsequently, the freeze-dried product of the decellularized pig kidney was further crushed by a crusher to obtain a crushed product of the decellularized pig kidney (K-dECM).

### Preparation of Gelling Agent

Subsequently, 100 mg of pepsin (2000 U/mg or more and 3000 U/mg or less) and 100 mL of 0.01 M HCl were added to 1 g of the crushed product of the decellularized pig kidney (K-dECM), the mixture was stirred at room temperature of 25° C. for 72 hours, and K-dECM was dissolved to obtain a gelled product. Subsequently, 0.1 M NaOH (⅒ amount of gelled product by mass ratio) and 10× PBS (pH 7.4) (⅑ amount of gelled product by mass ratio) were added to the gelled product at 4° C. to inactivate pepsin. Furthermore, the volume, concentration, and pH (pH 7.4) were adjusted, and the adjusted K-dECM gel was stored at 4° C.

### Example 3

### Preparation of Kidney Regeneration Accelerator (L-dECM Gel) Containing Decellularized Component Derived From Liver

### Collection and Storage of Pig Liver

After intravenous injection of heparin (5000 IU) into a pig (Göttingen minipig), the liver was mobilized and the gallbladder was resected. Subsequently, the bile duct, the hepatic artery, and the lower inferior vena cava were ligated, and then the liver was excised. The portal vein and the upper inferior vena cava were cannulated, and perfusion with physiological saline from the portal vein was performed until blood was no longer drained. After the perfusion, the excised liver was freeze-preserved at -80° C. in a state of being immersed in physiological saline.

### Decellularization by Stirring in Liquid

The freeze-preserved liver was slowly thawed at 4° C. Subsequently, using the thawed liver, stirring in PBS was performed for about 12 hours. Subsequently, it was immersed with PBS and left overnight. Subsequently, after stirring in PBS for about 1 hour, it was stirred in a 0.5 w/v% SDS aqueous solution for about 8 hours. Subsequently, after stirring in PBS for 2 hours, it was immersed with PBS and left overnight. Subsequently, after stirring in PBS for about 1 hour, it was stirred in PBS containing 0.5 v/v% Triton-X100, 0.05 w/v% EGTA, 0.05 w/v% sodium azide, and 2 mM CHAPS for about 8 hours. Subsequently, after stirring in PBS for 2 hours, it was immersed with PBS and left overnight. Subsequently, after stirring in PBS for about 10 hours, it was immersed in PBS and left overnight. Subsequently, it was stirred in PBS containing 25 µg/mL Anti-Anti and 0.3 mg/mL colistin for about 0.5 hours. Subsequently, it was stirred in PBS containing 25 µg/mL Anti-Anti and 1 w/v% gentamicin for about 0.5 hours to obtain a decellularized pig liver. Subsequently, the decellularized pig liver was shredded into about 8 mm square pieces. Subsequently, the decellularized pig liver shredded product was lightly dehydrated by centrifugation, frozen at -80° C., and then freeze-dried. Subsequently, the freeze-dried product of the decellularized pig liver was further crushed by a crusher to obtain a crushed product of the decellularized pig liver (L-dECM).

### Preparation of Gelling Agent

Subsequently, 100 mg of pepsin (2000 U/mg or more and 3000 U/mg or less) and 100 mL of 0.01 M HCl were added to 1 g of the crushed product of the decellularized pig liver (L-dECM), the mixture was stirred at room temperature of 25° C. for 72 hours, and L-dECM was dissolved to obtain a gelled product. Subsequently, 0.1 M NaOH (⅒ amount of gelled product by mass ratio) and 10× PBS (pH 7.4) (⅑ amount of gelled product by mass ratio) were added to the gelled product at 4° C. to inactivate pepsin. Furthermore, the volume, concentration, and pH (pH 7.4) were adjusted, and the adjusted L-dECM gel was stored at 4° C.

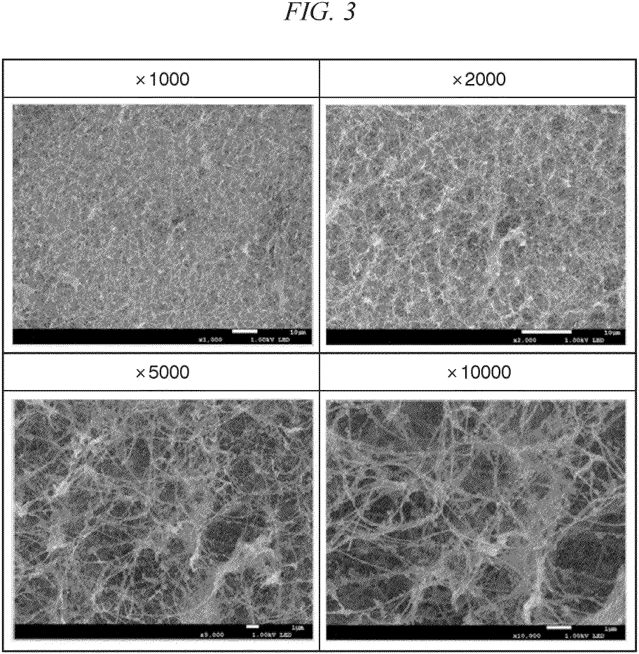

The dECM gel was prepared to 8 mg/mL, and then observed by a cryo-scanning electron microscope (cryo-SEM) under observation conditions of an acceleration voltage of 1.0 kV, a detector LED (secondary electron image), and a sample table temperature of around -90° C.). FIG. 3 is an SEM image of a kidney regeneration accelerator (L-dECM gel) containing a component derived from pig liver.

As shown in FIG. 3, it was confirmed that the dECM gel retained a network skeleton structure.

### Example 4

### Decellularization of Rat Kidney by Hydrostatic Pressure Treatment

Frozen rat kidney was left at room temperature for about 10 minutes and then thawed using a water bath at 37° C. The thawed kidney was perfused with PBS for about 2 hours, and then set in an isostatic pressing device. The pressure was increased to 500 MPa, maintained for 10 minutes, and then unloaded to 100 MPa. Then, the pressure was increased again, and the cycle of 500 MPa (10 minutes) → 100 MPa was repeated twice. Then, the pressure was returned to normal pressure and the kidney was taken out. Subsequently, the kidney was perfused with PBS for about 2 hours until the waste liquid became clear (flow rate of about 2 mL/min). Subsequently, 0.1 w/v% SDS aqueous solution perfusion was performed for about 12 hours (flow rate of about 0.2 mL/min). After perfusion with PBS for about 1 hour (flow rate of about 1 mL/min), the kidney was fixed by immersion in PBS containing 4 v/v% paraformaldehyde (PFA). It was confirmed that the rat kidney was well decellularized by visual observation and microscopic observation after HE staining.

### Test Example 2

### Injection Test of dECM Gel Into Rat Kidney

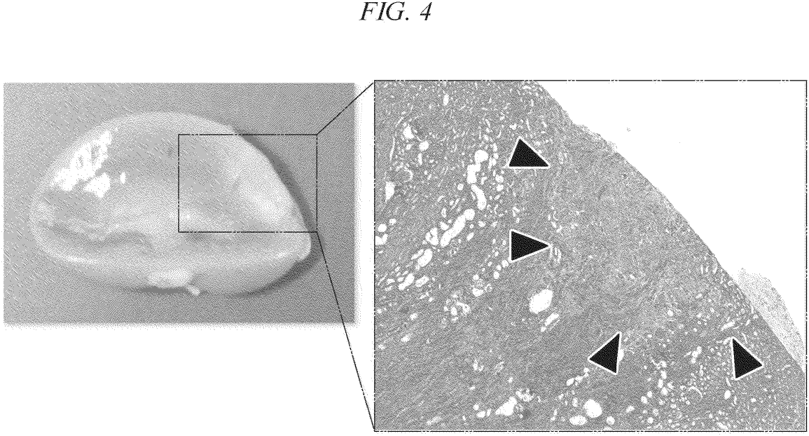

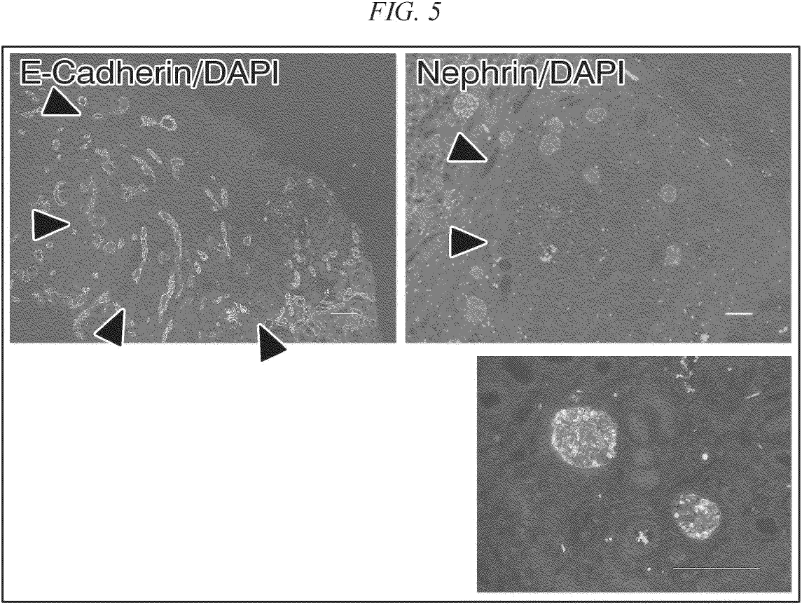

First, the abdomen of the rat was incised under isoflurane anesthesia to expose the kidney. Subsequently, partial nephrectomy was performed using scissors and tweezers. Subsequently, after compression hemostasis was performed, the L-dECM gel prepared in Example 3 was injected into the resection scar using an outer needle of a surflo indwelling needle and a syringe. Subsequently, it was left for a while until the surface of the injected L-dECM gel was solidified. It was confirmed that the L-dECM gel was thickened and lost its fluidity in about 5 minutes under the body temperature of the rat. Subsequently, the kidney was returned to the abdominal cavity and sutured. The gel-injected rat was normally bred for about 1 week from the injection test. Subsequently, under isoflurane anesthesia, the abdomen of the rat after the injection test was incised to excise the kidney. Rats after nephrectomy were euthanized. Subsequently, the excised kidney was fixed with PBS containing 4 v/v% PFA. Paraffin sections were prepared and subjected to HE staining and immunostaining using various antibodies (anti-E-cadherin antibody or anti-nephrin antibody). For the immunostained sections, nuclear staining was also performed using 4′,6-diamidino-2-phenylindole (DAPI). Images of the sections subjected to each staining were acquired with a microscope manufactured by KEYENCE CORPORATION. FIG. 4 is an observation image and an HE stained image of the excised rat kidney. FIG. 5 is a fluorescence stained image of the excised rat kidney.

As shown in FIG. 4, cell infiltration was observed in a case where the L-dECM gel was transplanted into the defective part of rat kidney (see the arrowhead in FIG. 4). Furthermore, as shown in FIG. 5, it was observed that renal tubules and glomeruli were regenerated (see the arrowhead in FIG. 5).

### Test Example 3

### Injection Test of dECM Gel into Pig Kidney

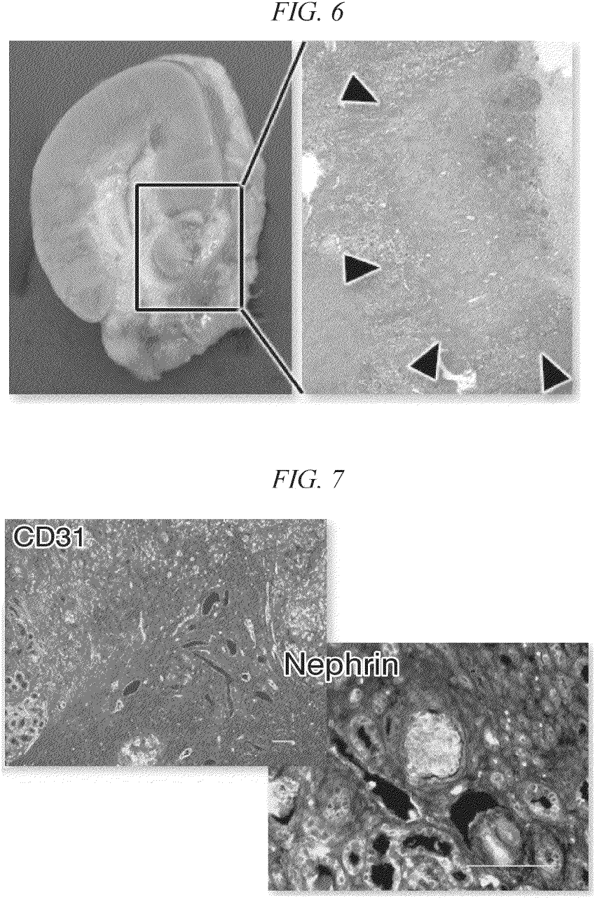

A midline incision was made in the pig under isoflurane anesthesia to expose the kidney. Subsequently, partial nephrectomy was performed using an electric knife and scissors. Subsequently, after coagulation hemostasis was performed with an electric knife, the L-dECM gel prepared in Example 3 was injected using the outer needle of the surflo indwelling needle and the syringe. It was left for a while until the surface of the injected L-dECM gel was solidified (about 5 minutes). Subsequently, after applying a sepra film to prevent adhesions, the Gerota fascia was sutured and the abdomen was closed. The gel-injected pig was normally bred for about 1 month from the injection test. Subsequently, under isoflurane anesthesia, a midline incision was made in the pig after the injection test to excise the kidney. Pigs after nephrectomy were euthanized by hemorrhagic death. Subsequently, the excised kidney was fixed with PBS containing 4 v/v% PFA. Paraffin sections were prepared and subjected to HE staining and immunostaining using various antibodies (anti-CD31 antibody, anti-nephrin antibody). Images of the sections subjected to each staining were acquired with a microscope manufactured by KEYENCE CORPORATION. FIG. 6 is an observation image and an HE stained image of the excised pig kidney. FIG. 7 is a fluorescence stained image of the excised pig kidney.

As shown in FIG. 6, cell infiltration was also observed in a case where the L-dECM gel was transplanted into the defective part of pig kidney (see arrowhead in FIG. 6). Furthermore, as shown in FIG. 7, it was also observed that vascular endothelium and glomeruli were regenerated.

### Example 5

### Preparation of dECM with Different Concentrations

The K-dECM gel obtained by the same method as in Example 2 was frozen at -80° C. and freeze-dried. The freeze-dried product was crushed by a crusher and sterilized by irradiating with 3 kGy gamma rays. Pure water was added thereto to obtain 8 mg/mL and 16 mg/mL K-dECM gels. The viscosities were measured with an EMS viscometer (measurement temperature of 25° C., rotation speed of 1000 rpm). The viscosity of the 8 mg/mL K-dECM gel was 30 mPa·s, and the viscosity of 16 mg/mL K-dECM gel was 350 mPa·s.

### Test Example 4

### Injection Test of dECM Gels Having Different Concentrations into Rat Kidney

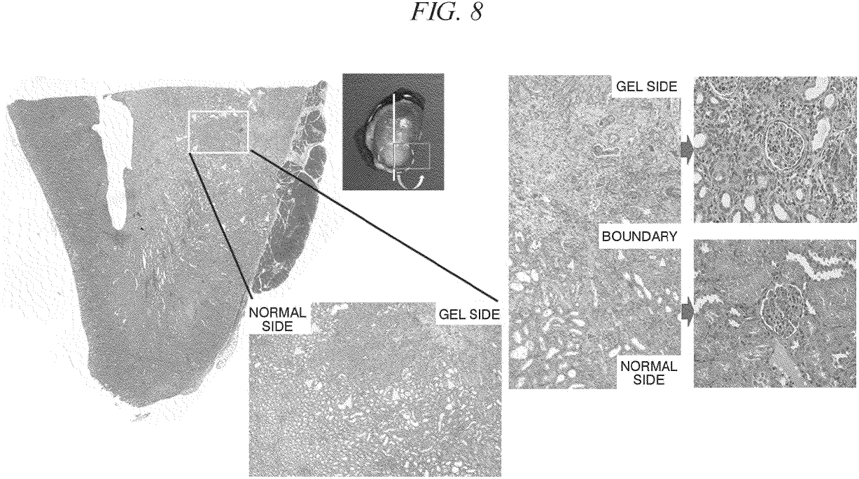

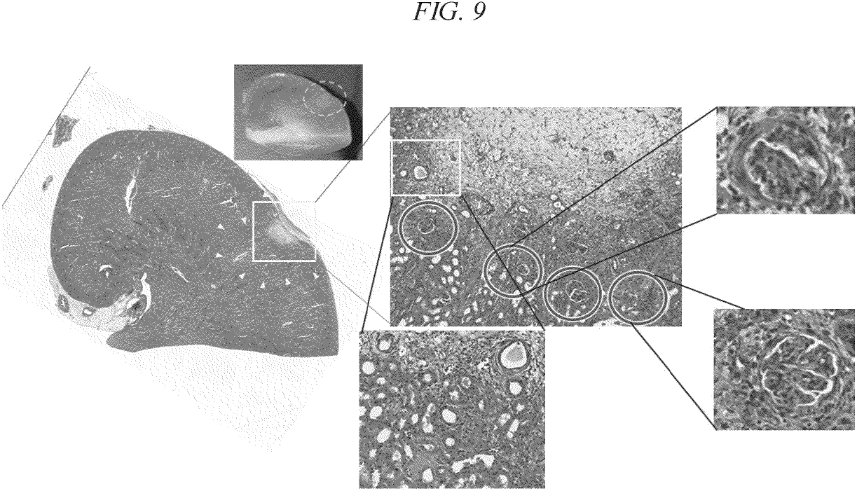

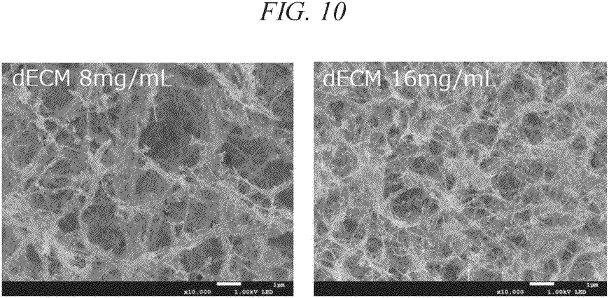

First, the abdomen of the rat was incised under isoflurane anesthesia to expose the kidney. Subsequently, partial nephrectomy was performed using scissors and tweezers. Subsequently, after compression hemostasis was performed, two types of K-dECM gels having different concentrations prepared in Example 5 were each injected into the resection scar using an outer needle of a surflo indwelling needle and a syringe. Subsequently, it was left for a while until the surface of the injected K-dECM gel was solidified. Subsequently, the kidney was returned to the abdominal cavity and sutured. The gel-injected rat was normally bred for about 1 week from the injection test. Subsequently, under isoflurane anesthesia, the abdomen of the rat after the injection test was incised to excise the kidney. Rats after nephrectomy were euthanized. Subsequently, the excised kidney was fixed with PBS containing 4 v/v% PFA. Paraffin sections were prepared and subjected to HE staining. Images of the HE stained sections were acquired with a microscope manufactured by KEYENCE CORPORATION. FIG. 8 (8 mg/mL K-dECM gel) and FIG. 9 (16 mg/mL K-dECM gel) are both observation images and HE stained images of the excised rat kidneys.

As shown in FIGS. 8 and 9, cell infiltration was observed in a case where any concentrations of K-dECM gels were used, but it was confirmed that in the 16 mg/mL K-dECM gel, cells infiltrated to more inside as compared with 8 mg/mL K-dECM gel (see arrowheads in FIGS. 8 and 9).

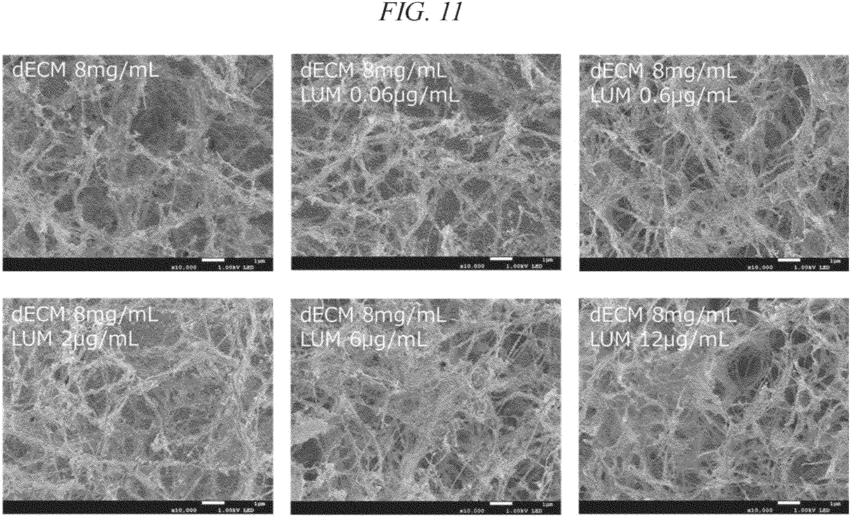

Furthermore, after the preparation, the K-dECM gels having different concentrations (8 mg/mL and 16 mg/mL) prepared in Example 5, were observed by a cryo-SEM under observation conditions of an acceleration voltage of 1.0 kV, a detector LED (secondary electron image), and a sample table temperature of around -90° C. FIG. 10 is cryo-SEM images of K-dECM gels having different concentrations (8 mg/mL and 16 mg/mL) prepared in Example 5.

From the comparison of FIGS. 8 and 9, and from FIG. 10, it was confirmed that the 16 mg/mL K-dECM gel formed a denser network structure than the 8 mg/mL K-dECM gel.

### Example 6

### Preparation of dECM with Various Amounts of Proteoglycan Added

An 8 mg/mL K-dECM gel was prepared using the same method as in Example 2. When adjusting a volume, a concentration, and pH (pH 7.4) of the 8 mg/mL K-dECM gel, proteoglycan was added such that the concentration of proteoglycan was 0, 0.06, 0.6, 2, 6, or 12 µg/mL in the total volume of the K-dECM gel, to obtain 6 types of K-dECM gels in which various amounts of proteoglycan were added.

After the preparation, each K-dECM gel obtained was observed by a cryo-SEM (magnification: 10000 times) under observation conditions of an acceleration voltage of 1.0 kV, a detector LED (secondary electron image), and a sample table temperature of around -90° C. FIG. 11 is cryo-SEM images of K-dECM gels in which the various amounts of proteoglycan were added.

As shown in FIG. 11, it was confirmed that the denser the network structure was formed as the concentration of proteoglycan increased.

### INDUSTRIAL APPLICABILITY

The kidney regeneration accelerator of the present embodiment is effective for the treatment of kidney disease.

1. A kidney regeneration accelerator comprising a component obtained by decellularizing an organ of a mammal.

2. The kidney regeneration accelerator according to claim 1,
wherein the kidney regeneration accelerator is selected from the group consisting of a solution, a dispersion, and a gel containing the component.

3. The kidney regeneration accelerator according to claim 2,
wherein a concentration of the component is 5 mg/mL or more and 25 mg/mL or less in a total volume of the kidney regeneration accelerator.

4. The kidney regeneration accelerator according to claim 2,
wherein a viscosity is 10 mPa·s or more and 1000 mPa s or less.

5. The kidney regeneration accelerator according to claim 1,
wherein the kidney regeneration accelerator is a powder.

6. The kidney regeneration accelerator according to claim 1,
wherein the component is obtained by decellularization including hydrostatic pressure treatment.

7. The kidney regeneration accelerator according to claim 1,
wherein the organ is one or more organs selected from the group consisting of a liver, a kidney, a spleen, a lung, a pancreas, an intestine, and a blood vessel.

8. A method of decellularizing an organ of a mammal to prepare a component containing an extracellular matrix, the method comprising:
performing hydrostatic pressure treatment on the organ or a fragment of the organ; and
perfusing the organ with water or stirring the fragment in water,
wherein the hydrostatic pressure treatment includes
pressurization including a positive pressure change in which an absolute value of an amount of the change is 100 MPa or more, and
decompression including a negative pressure change in which an absolute value of an amount of the change is 50 MPa or more each of which is alternately performed two times or more, and
the pressurization and the decompression are both performed at a pressure of 0 MPaG or more.

9. The method according to claim 8,
wherein the hydrostatic pressure treatment is performed in a state where the organ is in contact with a surfactant or a solution containing the surfactant.

10. A production method for a kidney regeneration accelerator, comprising:
decellularizing an organ of a mammal to obtain a component containing an extracellular matrix;
freeze drying and pulverizing the component to obtain a powder; and
performing sterilization treatment on the powder.

11. The production method for a kidney regeneration accelerator according to claim 10,
wherein the freeze drying is performed two times or more.

12. The production method for a kidney regeneration accelerator according to claim 10,
wherein the obtaining the component containing the extracellular matrix includes:
performing hydrostatic pressure treatment on the organ or a fragment of the organ; and
perfusing the organ with water or stirring the fragment in water.

13. The production method for a kidney regeneration accelerator according to claim 12,
wherein the hydrostatic pressure treatment includes
pressurization including a positive pressure change in which an absolute value of an amount of the change is 100 MPa or more, and
decompression including a negative pressure change in which an absolute value of an amount of the change is 50 MPa or more each of which is alternately performed two times or more, and
the pressurization and the decompression are both performed at a pressure of 0 MPaG or more.

14. The production method for a kidney regeneration accelerator according to claim 12,
wherein the hydrostatic pressure treatment is performed in a state where the organ is in contact with a surfactant or a solution containing the surfactant.

15-19. (canceled)

20. A treatment method for kidney disease, comprising:
applying a pharmaceutical composition containing a component obtained by decellularizing an organ of a mammal to a site to be treated of a kidney of a human or animal kidney disease patient.

21. The treatment method for kidney disease according to claim 20,
wherein decellularizing the organ of the mammal to obtain the component includes:
performing hydrostatic pressure treatment on the organ or a fragment of the organ; and
perfusing the organ with water or stirring the fragment in water,
the hydrostatic pressure treatment includes
pressurization including a positive pressure change in which an absolute value of an amount of the change is 100 MPa or more, and
decompression including a negative pressure change in which an absolute value of an amount of the change is 50 MPa or more each of which is alternately performed two times or more, and
the pressurization and the decompression are both performed at a pressure of 0 MPaG or more.

22. The treatment method for kidney disease according to claim 21,
wherein the hydrostatic pressure treatment is performed in a state where the organ is in contact with a surfactant or a solution containing the surfactant.

23. The treatment method for kidney disease according to claim 20,
wherein decellularizing an organ of a mammal to obtain the component includes:
performing hydrostatic pressure treatment on the organ or a fragment of the organ; and
perfusing the organ with water or stirring the fragment in water.

24. The treatment method for kidney disease according to claim 23,
wherein the hydrostatic pressure treatment includes
pressurization including a positive pressure change in which an absolute value of an amount of the change is 100 MPa or more, and
decompression including a negative pressure change in which an absolute value of an amount of the change is 50 MPa or more each of which is alternately performed two times or more, and
the pressurization and the decompression are both performed at a pressure of 0 MPaG or more.

25. The treatment method for kidney disease according to claim 23,
wherein the hydrostatic pressure treatment is performed in a state where the organ is in contact with a surfactant or a solution containing the surfactant.
